(19) United States
(12) Patent Application Publication
(10) Pub. No.: US 2025/0303088 A1
(43) Pub. Date: Oct. 2, 2025

(54) NASOPHARYNGEAL AIRWAY DEVICES

(71) Applicant: REGENTS OF THE UNIVERSITY OF MICHIGAN, Ann Arbor, MI (US)

(72) Inventors: David Adam Zopf, Ann Arbor, MI (US); Jeffrey Stephen Plott, Algonac, MI (US)

(21) Appl. No.: 18/863,928

(22) PCT Filed: Apr. 27, 2023
(86) PCT No.: PCT/US2023/020188

§ 371 Date: Nov. 7, 2024

Related U.S. Application Data
(60) Provisional application No. 63/341,911, filed on May 13, 2022.

(51) Int. Cl. A61M 16/04

(52) U.S. Cl. CPC A61M 16/0461; A61M 16/0431; A61M 16/0497; A61M 2209/088; A61M 2210/0618

(57) ABSTRACT

In an implementation, a nasopharyngeal airway device (100) includes an elongate body (102) having a distal end (104) and a proximal end (106). The elongate body defines a channel (108) extending along the elongate body. The elongate body has a wall (110) having a first portion (112) and a second portion (114). The first portion is thicker than the second portion to deter the elongate body from inwardly collapsing.

### RELATED APPLICATION

This application claims the benefit of and priority to U.S. Provisional Patent Application No. 63/341,911, filed May 13, 2022, the content of which is incorporated by reference herein in its entireties and for all purposes.

### FIELD OF THE DISCLOSURE

The application relates generally to nasopharyngeal airway devices.

### BACKGROUND

Obstructive sleep apnea is a sleeping disorder during which breathing stops and starts due to occlusion of the airway. Some known sleep apnea treatments include providing an individual with a continuous positive airway pressure mask that insufflates the airway to stay open through the delivery of pressurized air. These masks are bulky, noisy, and uncomfortable, and compliance is low.

Nasopharyngeal airway devices are used in some medical applications to maintain patency of the airway of an individual to stay open. The conventional nasopharyngeal airway device may be inserted into the nose of the individual and provides a tube through which air can flow. Insertion of such a device may be uncomfortable, and mucus may have a tendency to occlude the tube of the device.

### SUMMARY

This disclosure relates to open-channeled nasopharyngeal airway devices. The airway devices may be adapted to prevent mucus build-up. The airway devices disclosed may be tolerated in an indwelling condition for a longer duration than a tubular airway device as a result. The open channels of the disclosed airway devices may disperse the air flow in a manner that deters the individual using the airway devices from obtaining a sore throat. The open channels of the airway devices may also be easier to clean than a tubular airway device. The open channels of the airway devices may be adapted to resist occlusion if the airway device is kinked or compressed.

In accordance with a first implementation, a nasopharyngeal airway device includes an elongate body having a distal end and a proximal end. The elongate body define a channel extending along the elongate body. The elongate body has a wall having a first portion and a second portion. The first portion being thicker than the second portion to deter the elongate body from inwardly collapsing.

In accordance with a second implementation, a nasopharyngeal airway device includes an elongate body having a distal end and a proximal end. The elongate body defines a channel extending along the elongate body. The elongate body has a wall having a first portion and a second portion. The first portion having a higher material stiffness than the second portion to deter the elongate body from inwardly collapsing.

In accordance with a third implementation, a nasopharyngeal airway device includes an elongate body and a bridal securement component. The elongate body has a distal end and a proximal end. The elongate body defines a channel extending along the elongate body. The bridal securement component is securely coupled to the elongate body. The bridal securement component is to interact with a nasal bridal to secure the nasopharyngeal airway device within a nostril of an individual.

In accordance with a fourth implementation, an apparatus includes a nasopharyngeal airway device and a continuous positive airway pressure device. The nasopharyngeal airway device has an elongate body having a distal end and a proximal end. The elongate body defines a channel extending along the elongate body. The elongate body to be positioned within a nostril of an individual. The continuous positive airway pressure device to be worn by the individual while the elongate body is positioned within the nostril of the individual.

In further accordance with the foregoing first, second, third, and/or fourth implementations, an apparatus and/or method may further include any one or more of the following:

In accordance with one example, the nasopharyngeal airway device includes a securement component securely coupled to the elongate body. The securement component being configured to secure the nasopharyngeal airway device within a nostril of an individual.

In accordance with another example, the securement component includes a projection.

In accordance with another example, the projection includes an arced arm having a distal foot and extending toward the distal end of the elongate body. A proximal end of the arm being securely coupled to the elongate body.

In accordance with another example, the projection and the elongate body form a clip.

In accordance with another example, the channel includes an open channel.

In accordance with another example, the channel extends along the elongate body from the distal end to the proximal end.

In accordance with another example, the first portion is positioned adjacent a transverse midline of the elongate body.

In accordance with another example, the first portion includes a pair of opposing inwardly extending protrusions.

In accordance with another example, the protrusions include ribs.

In accordance with another example, the ribs extend between the proximal end and the distal end.

In accordance with another example, the inwardly extending protrusions include transverse compression resistance elements.

In accordance with another example, the elongate body includes edges having opposing inward facing stops that define a lateral opening.

In accordance with another example, the stops have at least one of flat surfaces or corresponding contours that are positioned to abut one another.

In accordance with another example, the stops include radial compression resistance elements.

In accordance with another example, the elongate body further includes a pair of opposing indentations positioned between the corresponding protrusions and the stops.

In accordance with another example, the protrusions are configured to engage the stops to deter the elongate body from inwardly collapsing.

In accordance with another example, the distal end is tapered.

In accordance with another example, the channel includes a closed channel.

In accordance with another example, the first portion is positioned adjacent a transverse midline of the elongate body.

In accordance with another example, the first portion includes a pair of opposing inwardly extending protrusions.

In accordance with another example, the protrusions include ribs.

In accordance with another example, the ribs extend between the proximal end and the distal end.

In accordance with another example, the first portion is positioned adjacent a vertical midline of the elongate body.

In accordance with another example, the first portion includes a pair of opposing inwardly extending protrusions.

In accordance with another example, the protrusions include ribs.

In accordance with another example, the ribs extend between the proximal end and the distal end.

In accordance with another example, the first portion includes a pair of opposing inwardly extending second protrusions.

In accordance with another example, the second protrusions include second ribs.

In accordance with another example, the second ribs extend between the proximal end and the distal end.

In accordance with another example, the elongate body has an outer surface and a cross-section of the outer surface forms a circular segment.

In accordance with another example, the elongate body has an outer surface and a cross-section of the outer surface includes a plurality of flat surfaces.

In accordance with another example, the first portion is positioned adjacent a transverse midline of the elongate body.

In accordance with another example, the first portion is positioned adjacent a vertical midline of the elongate body.

In accordance with another example, the first portion has a Shore A hardness of approximately 50 A and the second portion has a Shore A hardness of approximately 40 A.

In accordance with another example, the first portion has a Shore A hardness of between approximately 10 A and approximately 70 A and the second portion has a Shore A hardness of between approximately 10 A and approximately 70 A.

In accordance with another example, the first portion has a Shore A hardness of between approximately 30 A and approximately 60 A and the second portion has a Shore A hardness of between approximately 30 A and approximately 60 A.

In accordance with another example, the elongate body includes a first longitudinal portion, a second longitudinal portion, and a third longitudinal portion. The second longitudinal portion is positioned between the first longitudinal portion and the third longitudinal portion. The first longitudinal portion and the third longitudinal portion each includes the first portion and the second portion. The second longitudinal portion having an inner surface that defines the channel and has a spiral protrusion.

In accordance with another example, the second longitudinal portion is to deter circumferential compression in a nasopharyngeal region of an individual.

In accordance with another example, the first longitudinal portion is to deter lateral compression in an intranasal region of an individual and the third longitudinal portion is to deter lateral compression in a pharyngeal region of the individual.

In accordance with another example, the elongate body has an inner surface that defines the channel and has a spiral protrusion.

In accordance with another example, the bridal securement component includes a projection.

In accordance with another example, the projection includes a pair of apertures.

In accordance with another example, the projection includes a post.

In accordance with another example, the elongate body has an inner surface that defines the channel and has one or more transverse protrusions.

In accordance with another example, the one or more transverse protrusions are continuous transverse protrusions.

In accordance with another example, the one or more transverse protrusions are discrete transverse protrusions.

### BRIEF DESCRIPTION OF THE DRAWINGS

FIG. 1 is an isometric view of an implementation of a nasopharyngeal airway device in accordance with an example of the present disclosure.

FIG. 2 is a cross-sectional isometric view of the nasopharyngeal airway device of FIG. 1 showing the channel, the first portion, and the second portion.

FIG. 3 is an isometric view of a portion of the nasopharyngeal airway device of FIG. 1 showing the distal end.

FIG. 4 is a cross-sectional isometric view of another implementation of another nasopharyngeal airway device showing a channel, a first portion, and a second portion.

FIG. 5 is an isometric view of another implementation of a nasopharyngeal airway device having a channel that is a closed channel.

FIG. 6 is a cross-sectional isometric view of another implementation of a nasopharyngeal airway device showing a channel, a first portion, and a second portion.

FIG. 7 is an isometric view of another implementation of a nasopharyngeal airway device having a channel that is a closed channel.

FIG. 8 is an isometric view of another implementation of a nasopharyngeal airway device having a channel that is a closed channel.

FIG. 9 is a cross-sectional isometric view of another implementation of a nasopharyngeal airway device showing a channel, a first portion, and a second portion.

FIG. 10 is a cross-sectional isometric view of another implementation of a nasopharyngeal airway device showing a channel, a first portion, and a second portion.

FIG. 11 is a cross-sectional isometric view of another implementation of a nasopharyngeal airway device showing a channel, a first portion, and a second portion.

FIG. 12 is a cross-sectional isometric view of another implementation of a nasopharyngeal airway device showing a channel, a first portion, and a second portion.

FIG. 13 is an isometric view of another implementation of a nasopharyngeal airway device showing the elongate body, the distal end, the proximal end, and the channel.

FIG. 14 is an isometric view of another implementation of a nasopharyngeal airway device showing the elongate body, the distal end, and the proximal end.

FIG. 15 is an isometric view of another implementation of a nasopharyngeal airway device showing the elongate body, the distal end, and the proximal end.

FIG. 16 is a schematic illustration showing an individual, a nasopharyngeal airway device, and a continuous positive airway pressure (CPAP) device.

FIG. 17 is an isometric view of another implementation of a nasopharyngeal airway device showing the elongate body, the distal end, the proximal end, and the channel.

### DETAILED DESCRIPTION

Although the following text discloses a detailed description of example methods, apparatus and/or articles of manufacture, it should be understood that the legal scope of the property right is defined by the words of the claims set forth at the end of this patent. Accordingly, the following detailed description is to be construed as examples only and does not describe every possible example, as describing every possible example would be impractical, if not impossible. Numerous alternative examples could be implemented, using either current technology or technology developed after the filing date of this patent. It is envisioned that such alternative examples would still fall within the scope of the claims.

Nasopharyngeal airways can be used to bypass upper airway obstructions. One common need for bypassing an upper airway obstruction is when a patient has obstructive sleep apnea caused by such upper airway obstruction. In the case of sleep apnea, these upper airway obstructions often result from the base of the tongue pressing against the back of the throat. Nasopharyngeal airway devices should be both efficacious in its ability to relieve and bypass the upper airway obstruction while also increasing the comfort for the patient when in use. In the case of a nasopharyngeal airway, these two elements, efficacy and comfort, can often work against each other. For example, a nasopharyngeal airway device with high stiffness and rigidity, such as an endotracheal tube, may be highly resistant to collapse under the forces of the upper airway and relieve obstructions well but may be uncomfortable for the patient to wear. On the other hand, a very soft and flexible nasopharyngeal airway device may be more comfortable for a patient to wear but may be more susceptible to collapse under the forces of the upper airway, causing the device to not relieve upper airway obstruction well. Another element for efficacy and comfort is the device size. For example, a device with greater effective diameter (or airway cross-sectional area) may provide greater efficacy at the cost of reduced comfort. It is generally preferable to maximize the available area for airflow while minimizing the overall profile of the device.

The implementations disclosed address these design tradeoffs by utilizing a design which has both high flexibility for comfort while also being highly resistant to total airway collapse under the forces of the upper airway obstruction, and therefor highly efficacious.

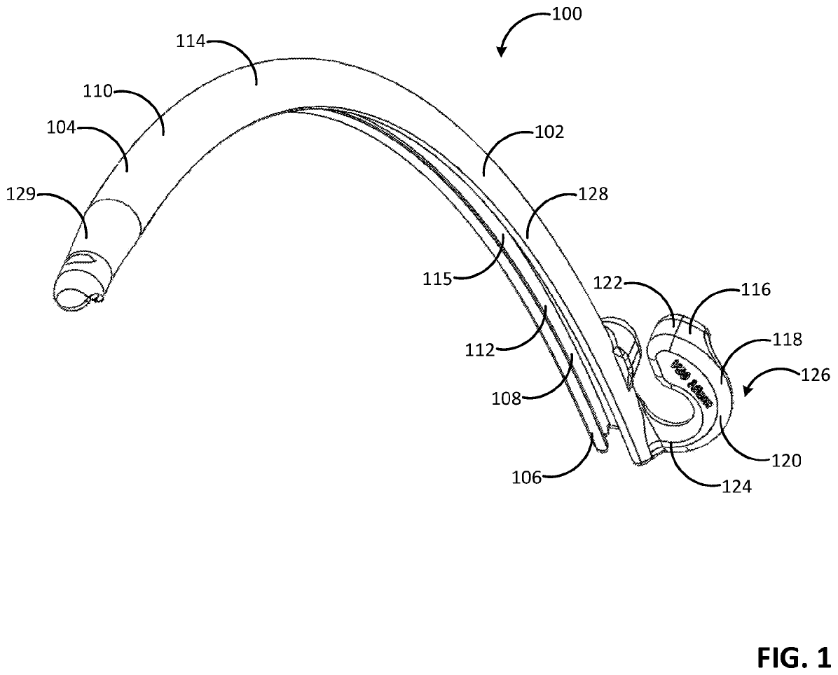

FIG. 1 is an isometric view of an implementation of a nasopharyngeal airway device 100 in accordance with an example of the present disclosure. The nasopharyngeal airway device 100 may be used for air-way patency through the nose. The nasopharyngeal airway device 100 is adapted to be worn the entire night while an individual is sleeping to prevent or reduce upper airway obstruction in some examples. The nasopharyngeal airway device 100 may thus be helpful to treat sleep apnea. The nasopharyngeal airway device 100 may be worn while the individual is awake. The nasopharyngeal airway device 100 may be helpful to assist respiration of children or other individuals with severe facial abnormalities and/or hypotonia, for example.

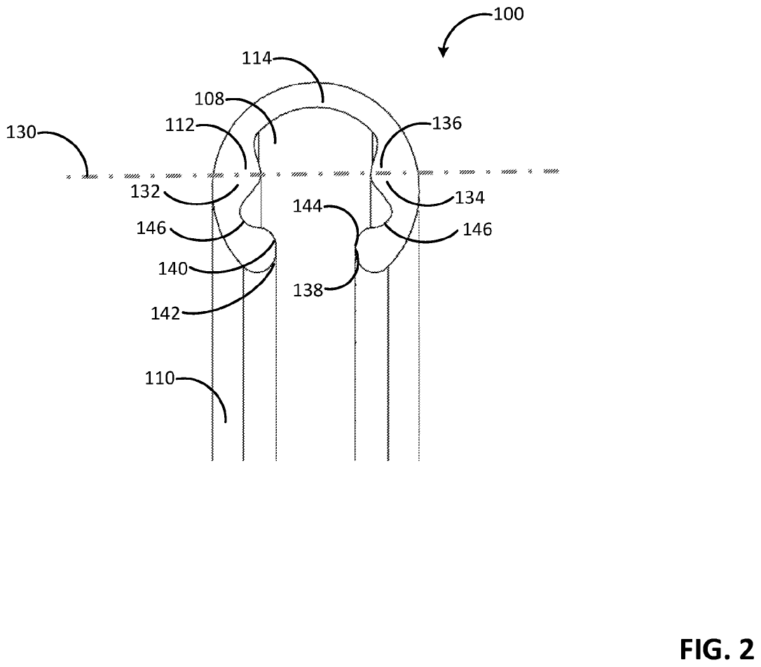

The nasopharyngeal airway device 100 includes an elongate body 102 in the example shown. The elongate body 102 may be referred to as a fluted body or an open-channel body. The elongate body 102 may have a general curvature to aid in the insertion of the elongate body 102 into the nose of the individual and more favorably turning caudally to bypass posterior to the palate rather than agitating the nasopharynx. The elongate body 102 has a distal end 104 and a proximal end 106. The elongate body 102 defines a channel 108 extending along the elongate body 102. The elongate body 102 has a wall 110 having a first portion 112 and a second portion 114 (the first and second portions 112, 114 are more clearly shown in FIG. 2). The first portion 112 is thicker than the second portion 114 to deter the elongate body 102 from inwardly collapsing. The channel 108 is an open channel 115 in the implementation shown that extends between the distal end 104 and the proximal end 106.

The nasopharyngeal airway device 100 also includes a securement component 116 securely coupled to the elongate body 102. The securement component 116 is configured to secure the nasopharyngeal airway device 100 within a nostril of an individual. The securement component 116 includes a projection 118 having an arced arm 120. The arced arm 120 has a distal foot 122 extending toward the distal end 104 of the elongate body 102 and a proximal end 124 securely coupled to the elongate body 102. The projection 118 and the elongate body 102 form a clip 126.

A length of the elongate body may be patient specific but may generally extend, by way of example only, between approximately 7 centimeters (cm) and approximately 17 cm and/or between approximately 5 cm and approximately 16 cm. The elongate body 102 may have different lengths such as for neonatal, pediatric, adolescent, and adult use as appropriate. An overall internal diameter of the elongate body 102 may be between approximately 2 mm and approximately 8 mm, for example. A portion 128 of the elongate body 102 may have a first radius of curvature and a second portion 129 adjacent the distal end 104 may have a second radius of curvature. The first radius of curvature may be, by way of example only, approximately 3.25 having a diameter of approximately 6.5 millimeters (mm) and the second radius of curvature may be approximately 2.75 mm having a diameter of approximately 5.5 mm. These curvature radii could vary depending on the needs of the patient (for example pediatric vs adult). The second portion 129 is shown being tapered toward the distal end 104 and as an atraumatic tip. The nasopharyngeal airway device 100 may pass along the floor of the nose, medial to the inferior and medial turbinates, and pass posterior to the soft palate with minimal agitation as the nasopharyngeal airway device 100 is inserted, due to the combination of the curvature of the nasopharyngeal airway device 100 and deflecting surface of the atraumatic tip, for example.

The nasopharyngeal airway device 100 including the elongate body 102 may be made from a medical grade silicone elastomer with Shore A hardness of between approximately 30 A and approximately 60 A. The nasopharyngeal airway device 100 including the elongate body 102 may be made from a medical grade silicone elastomer with Shore A hardness of between approximately 10 A and approximately 70 A as an example. The nasopharyngeal airway device 100 and the elongate body 102 may thus be relatively flexible and may be relatively easy to insert into the nostril/nasal cavity of the individual. Other materials and/or harnesses may be used.

The nasopharyngeal airway device 100 or any of the disclosed examples may be made of silicone, thermoplastic elastomer, PVC and may be produced using any technique such as injection molding, forming, three-dimensional (3D) printing, bonding multiple components together, and/or extrusion techniques. The proximal end 106 is a stiffer material such as Polyvinyl chloride (PVC) and the distal end 106 may be a softer material such as silicone in some implementations.

The nasopharyngeal airway device 100 may include a surface texture and/or a coating. The coating may be a lubricious coating (e.g., a water-based lubricant) that includes a local anesthetic coating such as Lidocaine, for example. The nasopharyngeal airway device 100 may be packaged in a foil pouch or in another fluid-resistant packaging in implementations where a lubricant and/or a local anesthetic are provided. The nasopharyngeal airway device 100 may alternatively be packaged in a Tyvek pouch, and the lubricant and/or the local anesthetic may be provided separately, for example.

FIG. 2 is a cross-sectional isometric view of the nasopharyngeal airway device 100 of FIG. 1 showing the channel 108, the first portion 112, and the second portion 114. The first portion 112 is thicker than the second portion 114, is positioned adjacent a transverse midline 130 of the elongate body 102, and includes a pair of opposing inwardly extending protrusions 132, 134 in the implementation shown. The protrusions 132, 134 may be referred to as transverse compression resistance elements. The first portion may be placed near the midline 130 so as to decrease the increase in bending stiffness of the nasopharyngeal airway device 100.

The protrusions 132, 134 are ribs 136 that extend between the proximal end 106 and the distal end 104. The ribs 136 may be spaced from the distal end 104 in some implementations. The ribs 136 may extend to the distal end 104 in other implementations. Alternatively, the ribs 136 may only encompass part of the channel 108. For example, they may only be included in specific areas of the airway under higher compression.

The elongate body 102 also includes edges 138 having opposing inward facing stops 140 that define a lateral opening 142. The stops 140 may be referred to as radial compression resistance elements. The stops 140 have flat surfaces or corresponding contours 144 that are positioned to abut one another. The elongate body 102 also includes a pair of opposing indentations 146 positioned between the protrusions 132, 134 and the stops 140. The indentations 146 may allow the protrusions 132, 134 and the stops 140 to move relative to and/or toward one another. The protrusions 132, 134 may thus be configured to engage the stops 140 to deter the elongate body 102 from inwardly collapsing. The engagement between the protrusions 132, 134 and the stops 140 may maintain a patent airway while also enabling device compliance which improves patient comfort.

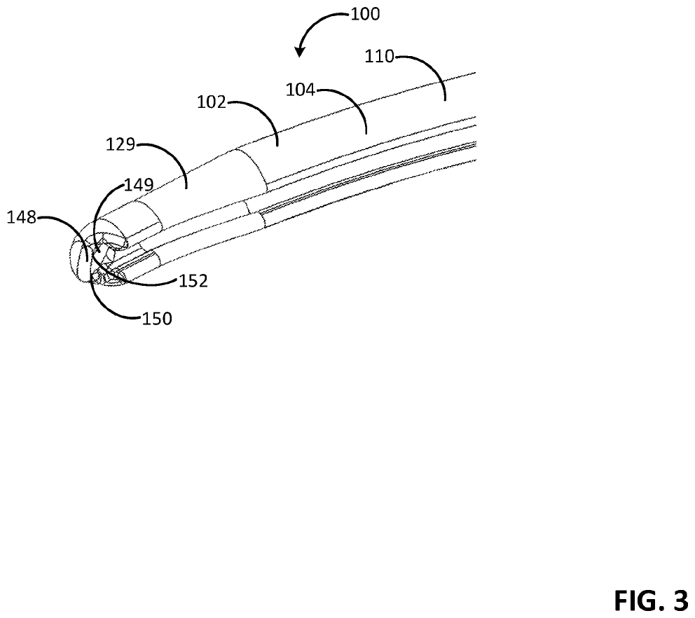

FIG. 3 is an isometric view of a portion of the nasopharyngeal airway device 100 of FIG. 1 showing the distal end 104. The distal end 104 of the nasopharyngeal airway device 100 includes a hood or partially closed rounded end 148 that eases insertion of the nasopharyngeal airway device 100 into the nasal airway of an individual. The closed rounded end 148 may be closed off to create a deflecting surface while another portion 149 of the distal end 104 is open to increase airflow and decrease clogging.

An inner surface 150 at the distal end 104 of the elongate body 102 defines a portion the channel 108 and forms a stop 152 that can be engaged by a suction device or an endoscope to prevent the suction device or scope from extending into the airway beyond the stop 152. This feature enables easy insertion with the hood while increasing the airway opening for device efficacy and clog prevention in some implementations.

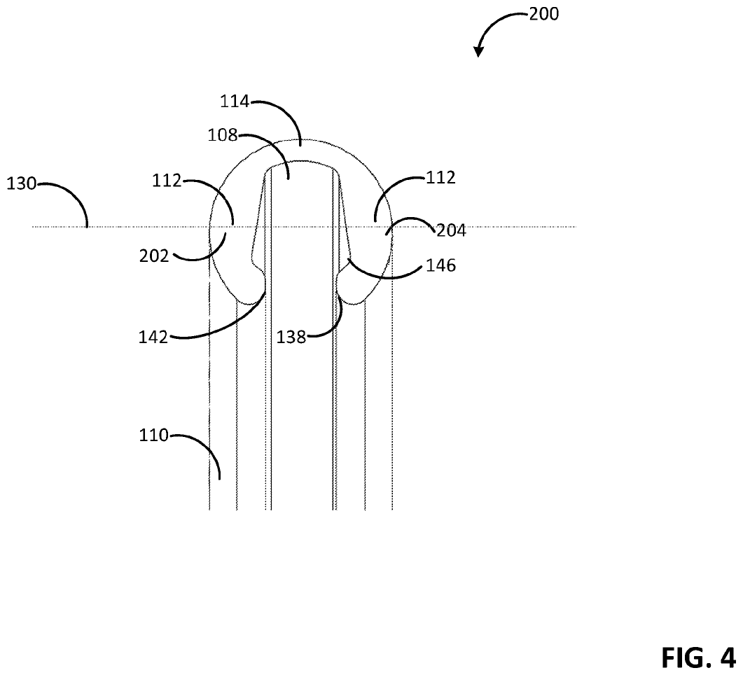

FIG. 4 is a cross-sectional isometric view of another implementation of a nasopharyngeal airway device 200 showing the channel 108, the first portion 112, and the second portion 114. The first portion 112 is shown as opposing portions 202, 204 of the nasopharyngeal airway device 200 that is thicker than the second portion 114. The indentations 146 are shown positioned between the opposing portions 202, 204 and the stops 140.

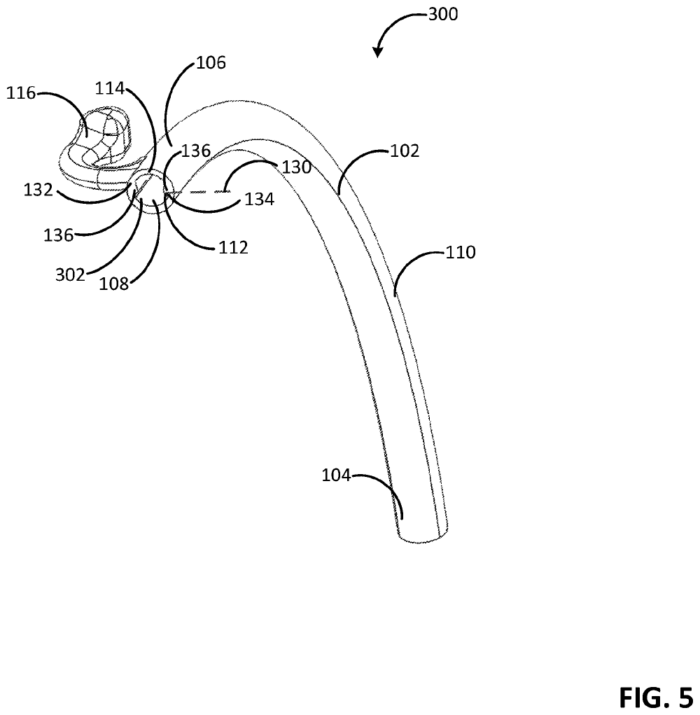

FIG. 5 is an isometric view of another implementation of a nasopharyngeal airway device 300 having the channel 108 that is a closed channel 302. The elongate body 102 of the nasopharyngeal airway device 300 has the first portion 112 that is thicker than the second portion 114 and is positioned adjacent the transverse midline 130 of the elongate body 102. The elongate body 102 of FIG. 5 also includes the pair of opposing inwardly extending protrusions 132, 134 in the implementation shown.

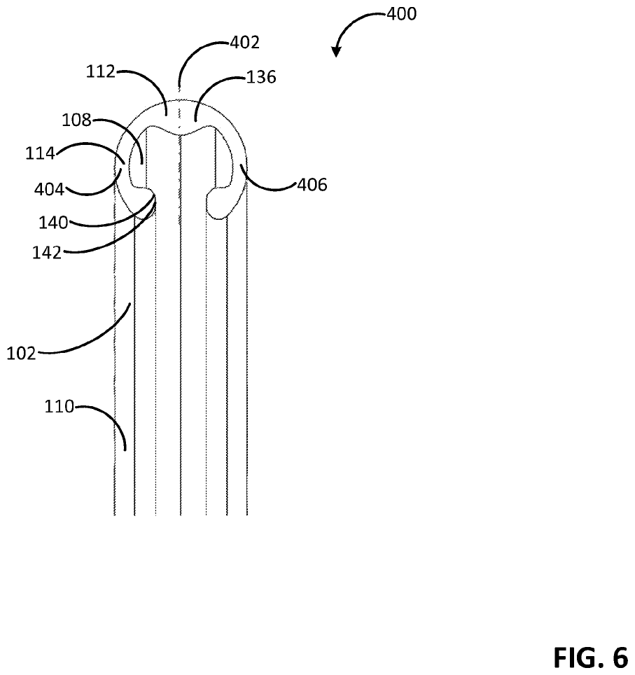

FIG. 6 is a cross-sectional isometric view of another implementation of a nasopharyngeal airway device 400 showing the channel 108, the first portion 112, and the second portion 114. The first portion 112 is positioned adjacent a vertical midline 402 of the elongate body 102. The nasopharyngeal airway device 400 may be beneficial in patients with lateral airway collapse. The second portion 114 is shown as opposing portions 404, 406 that are positioned between the first portion 112 and corresponding stops 140. The first portion 112 of FIG. 6 may be a rib 113 that extends between the distal end 104 and the proximal end 106.

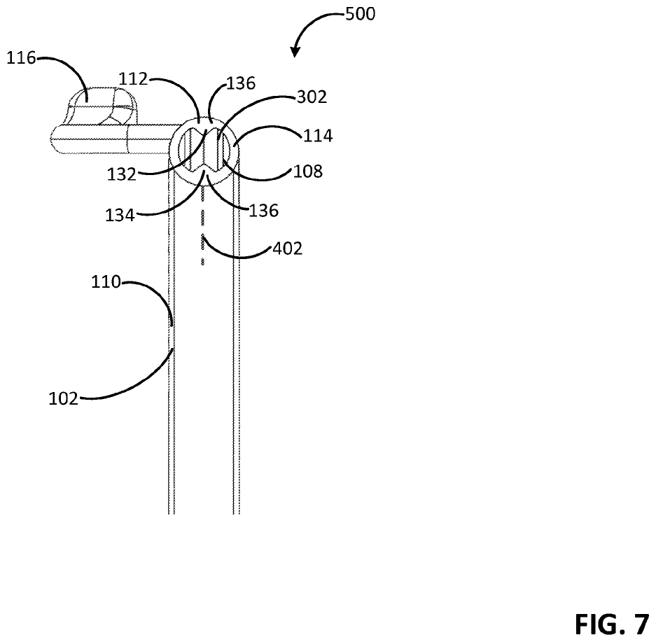

FIG. 7 is an isometric view of another implementation of a nasopharyngeal airway device 500 having the channel 108 that is the closed channel 302. The elongate body 102 of the nasopharyngeal airway device 300 has the first portion 112 that is thicker than the second portion 114 and is positioned adjacent the vertical midline 402 of the elongate body 102. The elongate body 102 of FIG. 7 also includes the pair of opposing inwardly extending protrusions 132, 134 in the implementation shown.

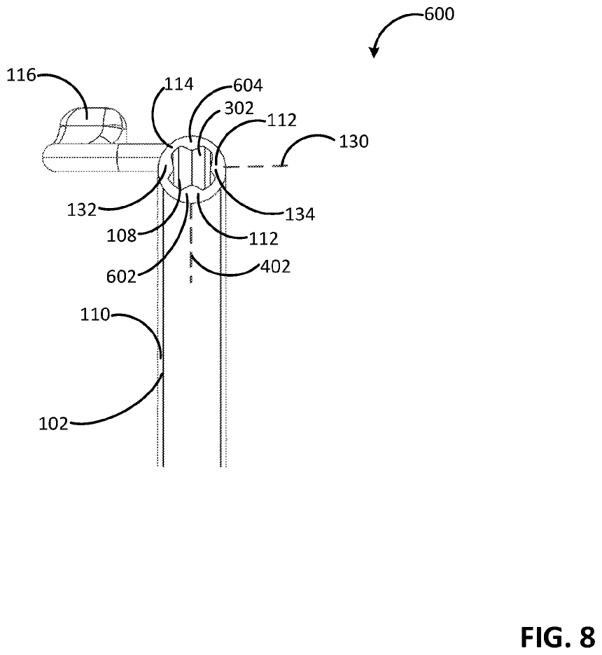

FIG. 8 is an isometric view of another implementation of a nasopharyngeal airway device 600 having the channel 108 that is the closed channel 302. The first portion 112 includes the pair of the opposing inwardly extending protrusions 132, 134 and a pair of opposing inwardly extending second protrusions 602, 604. The second protrusions 602, 604 are also shown as ribs 136 that may extend between the proximal end 106 and the distal end 104. The ribs 136 of the second protrusions 602, 604 may be referred to as second ribs.

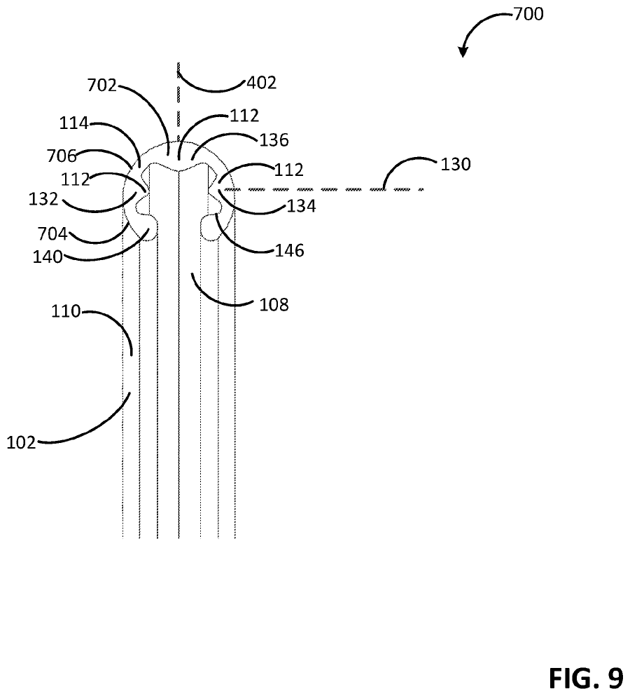

FIG. 9 is a cross-sectional isometric view of another implementation of a nasopharyngeal airway device 700 showing the channel 108, the first portion 112, and the second portion 114. The first portion 112 includes a plurality of the inwardly extending protrusions 132, 134, 702. The protrusions 132, 134 are positioned adjacent the transverse midline 130 and the protrusion 702 is positioned adjacent the vertical midline 402. The elongate body 102 of FIG. 9 has an outer surface 704 that has a cross-section that forms a circular segment 706. The outer surface 704 of the nasopharyngeal airway device 700 or any of the disclosed implementations may have a polished finish or a textured finish.

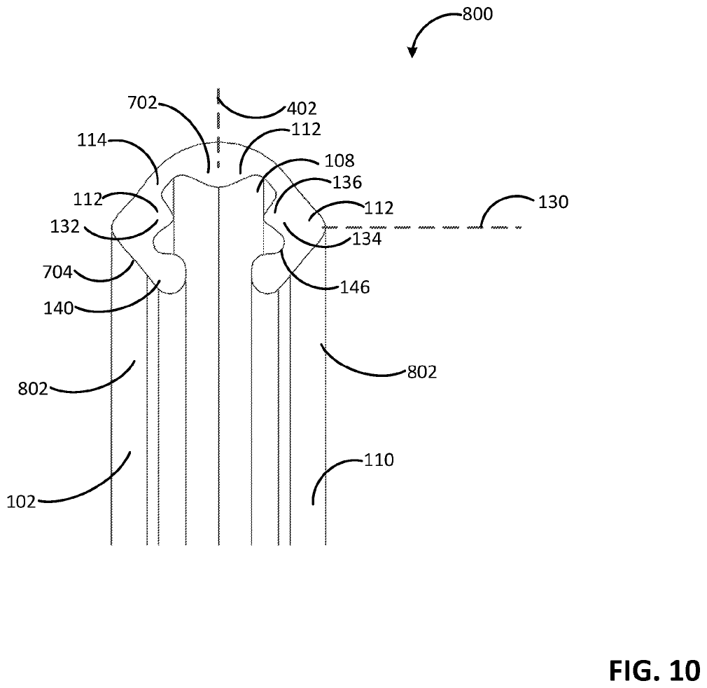

FIG. 10 is a cross-sectional isometric view of another implementation of a nasopharyngeal airway device 800 showing the channel 108, the first portion 112, and the second portion 114. The nasopharyngeal airway device 800 is similar to the nasopharyngeal airway device 700 of FIG. 9. The nasopharyngeal airway device 800 includes the outer surface 704 having a plurality of flat surfaces 802, however. The outer surface 704 may be alternative shapes such an oval-like shape.

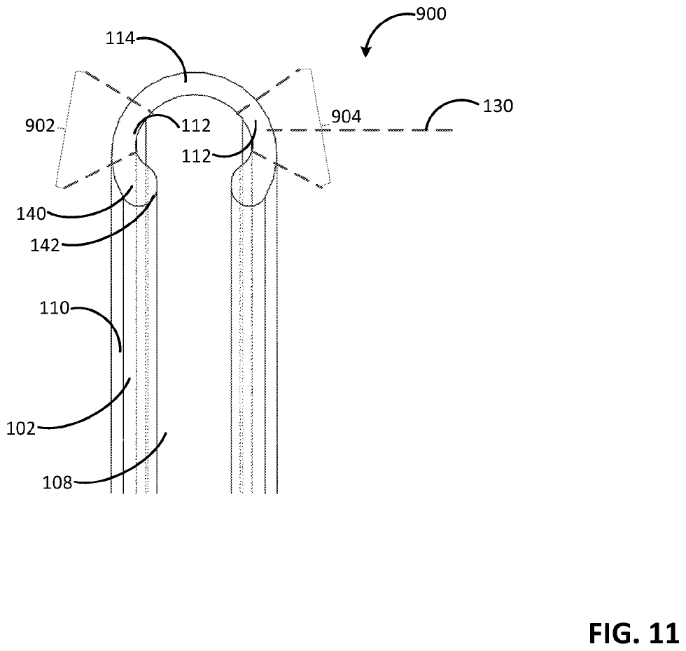

FIG. 11 is a cross-sectional isometric view of another implementation of a nasopharyngeal airway device 900 showing the channel 108, the first portion 112, and the second portion 114. The first portion 112 has a higher material stiffness than the second portion 114 to deter the elongate body 102 from inwardly collapsing. The first portion 112 is positioned adjacent the transverse midline 130 of the elongate body 102 in the implementation shown. The first portion 112 includes opposing portions 902, 904 of the elongate body 102. The first portion 112 may have a Shore A hardness of approximately 50 A and the second portion 114 may have a Shore A hardness of approximately 40 A. The first portion 112 may have a Shore A hardness of between approximately 10 A and approximately 70 A the second portion 114 may have a Shore A hardness of between approximately 10 A and approximately 70 A as an example. The first portion 112 may have a Shore A hardness of between approximately 30 A and approximately 60 A and the second portion 114 may have a Shore A hardness of between approximately 30 A and approximately 60 A as another example.

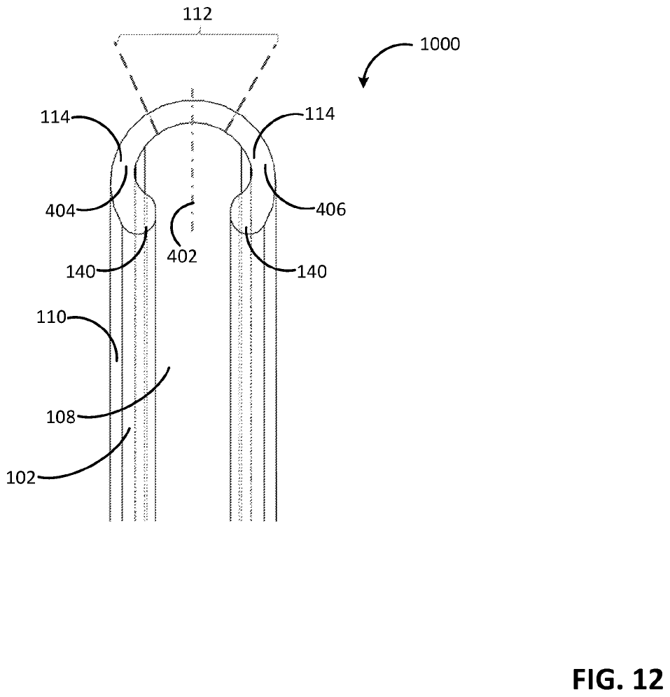

FIG. 12 is a cross-sectional isometric view of another implementation of a nasopharyngeal airway device 1000 showing the channel 108, the first portion 112, and the second portion 114. The first portion 112 has a higher material stiffness than the second portion 114 to deter the elongate body 102 from inwardly collapsing. The first portion 112 is positioned adjacent the vertical midline 402 of the elongate body 102. The second portion 114 is shown as opposing portions 404, 406 that are positioned between the first portion 112 and corresponding stops 140.

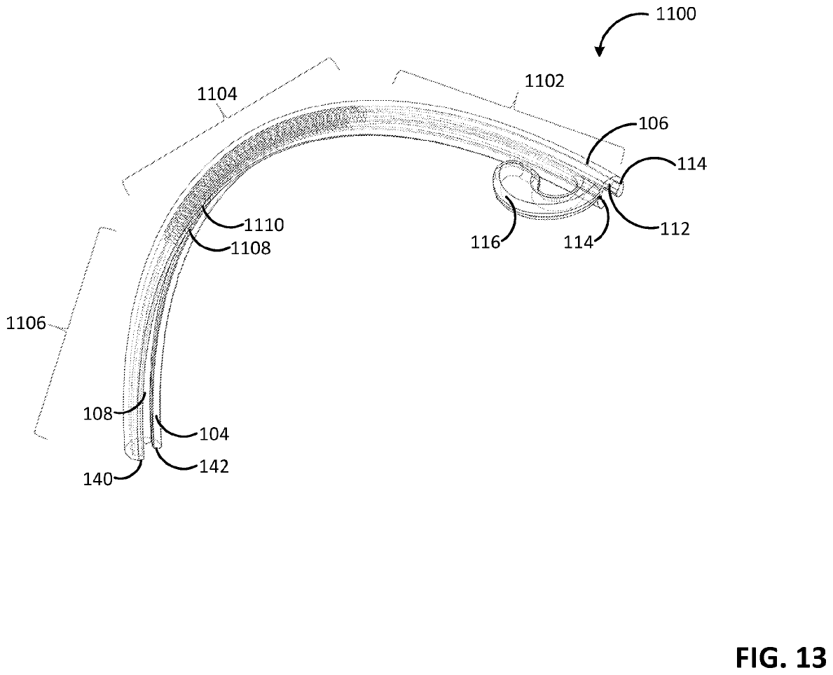

FIG. 13 is an isometric view of another implementation of a nasopharyngeal airway device 1100 showing the elongate body 102, the distal end 104, the proximal end 106, and the channel 108. The elongate body 102 includes a first longitudinal portion 1102, a second longitudinal portion 1104, and a third longitudinal portion 1106 in the implementation shown. The second longitudinal portion 1104 is positioned between the first longitudinal portion 1102 and the third longitudinal portion 1106 and the first longitudinal portion 1102 and the third longitudinal portion 1106 each include the first portion 112 and the second portion 114. The second longitudinal portion 1104 has an inner surface 1108 that defines the channel 108 and has a spiral protrusion 1110. The first longitudinal portion 1102 includes and/or is adjacent the proximal end 106 and the third longitudinal portion 1106 includes and/or is adjacent the distal end 104. The spiral protrusion 1110 may more generally be referred to a transverse protrusion and/or a continuous transverse protrusion.

The second longitudinal portion 1104 deters circumferential compression in a nasopharyngeal region of an individual using the nasopharyngeal airway device 1100. The first longitudinal portion 1102 deters lateral compression in an intranasal region of an individual wearing the nasopharyngeal airway device 1100 and the third longitudinal portion deters lateral compression in a pharyngeal region of the individual wearing the nasopharyngeal airway device 1100. Put another way, the first portion 112 and the second portion 114 deters lateral compression and the spiral protrusion 1110 deters circumferential compression.

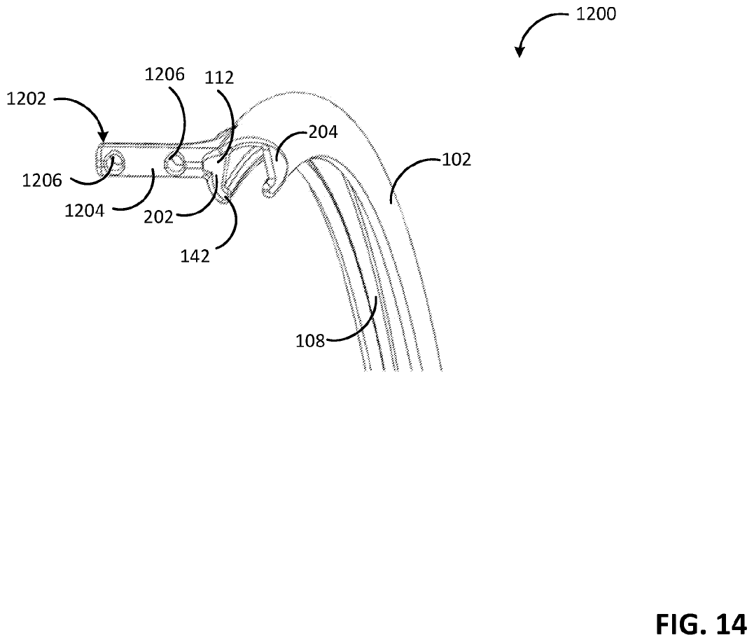

FIG. 14 is an isometric view of another implementation of a nasopharyngeal airway device 1200 showing the elongate body 102, the distal end 104, and the proximal end 106. The elongate body 102 defines the channel 108 extending along the elongate body 102. The nasopharyngeal airway device 1200 also includes a bridal securement component 1202 securely coupled to the elongate body 102. The bridal securement component 1202 interacts with a nasal bridal in operation to secure the nasopharyngeal airway device 1200 within a nostril of an individual. The bridal securement component includes a projection 1204 defining a pair of apertures 1206 in the implementation shown. A fastener of the nasal bridal may be threaded through the apertures 1206 to secure the nasal bridal to the projection 1204.

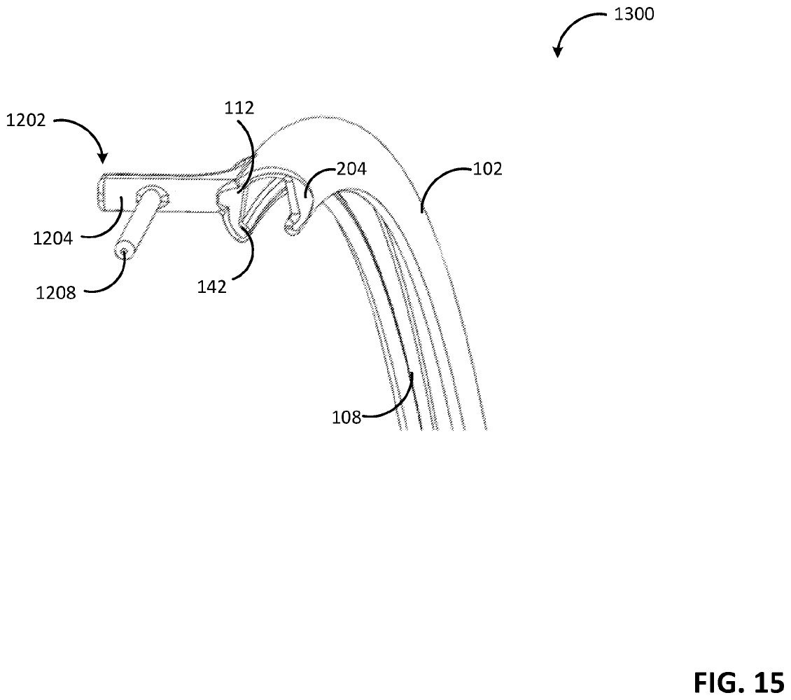

FIG. 15 is an isometric view of another implementation of a nasopharyngeal airway device 1300 showing the elongate body 102, the distal end 104, and the proximal end 106. The nasopharyngeal airway device 1300 of FIG. 15 is similar to the nasopharyngeal airway device 1200 of FIG. 14. The projection 1204 of the bridal securement component 1202 of FIG. 15 includes a post 1208, however. A fastener of the nasal bridal may be coupled around the post 1208 to secure the nasal bridal to the projection 1204.

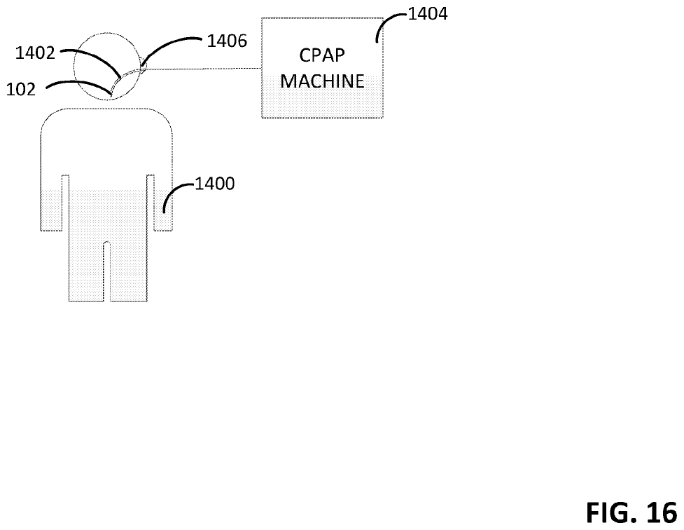

FIG. 16 is a schematic illustration showing an individual 1400, a nasopharyngeal airway device 1402, and a continuous positive airway pressure (CPAP) device 1404. The nasopharyngeal airway device 1402 of FIG. 16 is shown positioned within a nostril 1406 of the individual and may be implemented by any of the nasopharyngeal airway devices 100, 200, 300, 400, 500, 600, 700, 800, 900, 1000, 1100, 1200, 1300, and/or 1500 disclosed. The continuous positive airway pressure device 1404 is shown being worn by the individual 1400 while the elongate body 102 is positioned within the nostril 1406 of the individual 1400.

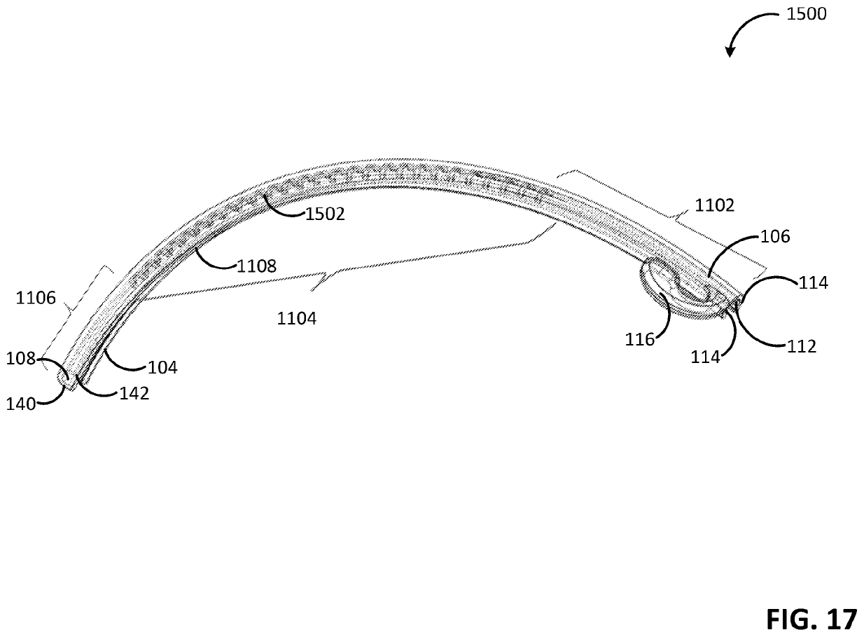

FIG. 17 is an isometric view of another implementation of a nasopharyngeal airway device 1500 showing the elongate body 102, the distal end 104, the proximal end 106, and the channel 108. The elongate body 102 includes the first longitudinal portion 1102, the second longitudinal portion 1104, and the third longitudinal portion 1106 in the implementation shown. The nasopharyngeal airway device 1500 is similar to the nasopharyngeal airway device 1100 of FIG. 13. The nasopharyngeal airway device 1500 includes transverse protrusions 1502 that are discrete, however. The transverse protrusions 1502 of FIG. 18 are thus not a spiral. The transverse protrusions 1502 may be referred to rings or ring segments. The rib(s) 136 may extend through all or a portion of the transverse protrusions 1502. The rib(s) 136 may alternatively be omitted from the second longitudinal portion 1104.

While several examples have been disclosed herein, any features from any examples may be combined with or replaced by other features from other examples. Moreover, while several examples have been disclosed herein, changes may be made to the disclosed examples without departing from the scope of the claims.

What is claimed is:

1. A nasopharyngeal airway device, comprising:
an elongate body having a distal end and a proximal end, the elongate body defining a channel extending along the elongate body, the elongate body comprising a wall having a first portion and a second portion, the first portion being thicker than the second portion to deter the elongate body from inwardly collapsing.

2. The nasopharyngeal airway device of claim 1, further comprising a securement component securely coupled to the elongate body, the securement component being configured to secure the nasopharyngeal airway device within a nostril of an individual, wherein the securement component includes a projection, wherein the projection comprises an arced arm having a distal foot and extending toward the distal end of the elongate body, a proximal end of the arm being securely coupled to the elongate body, wherein the projection and the elongate body form a clip.

3-6. (canceled)

6. The nasopharyngeal airway device of claim 1, wherein the channel comprises an open channel, wherein the channel extends along the elongate body from the distal end to the proximal end.

7. (canceled)

8. The nasopharyngeal airway device of claim 1, wherein the first portion is positioned adjacent a transverse midline of the elongate body, wherein the first portion comprises a pair of opposing inwardly extending protrusions.

9-12. (canceled)

13. The nasopharyngeal airway device of claim 1, wherein the elongate body includes edges having opposing inward facing stops that define a lateral opening.

14-15. (canceled)

16. The nasopharyngeal airway device of claim 13, wherein the elongate body further comprises a pair of opposing indentations positioned between the corresponding protrusions and the stops.

17-18. (canceled)

19. The nasopharyngeal airway device of claim 1, wherein the channel comprises a closed channel.

20. The nasopharyngeal airway device of claim 19, wherein the first portion is positioned adjacent a transverse midline of the elongate body, wherein the first portion comprises a pair of opposing inwardly extending protrusions.

21-23. (canceled)

24. The nasopharyngeal airway device of claim 1, wherein the first portion is positioned adjacent a vertical midline of the elongate body.

25. The nasopharyngeal airway device of claim 24, wherein the first portion comprises a pair of opposing inwardly extending protrusions.

26-27. (canceled)

28. The nasopharyngeal airway device of claim 24, wherein the first portion comprises a pair of opposing inwardly extending second protrusions.

29-30. (canceled)

31. The nasopharyngeal airway device of claim 1, wherein the elongate body has an outer surface and a cross-section of the outer surface forms a circular segment.

32. The nasopharyngeal airway device of claim 1, wherein the elongate body has an outer surface and a cross-section of the outer surface comprises a plurality of flat surfaces.

33. The nasopharyngeal airway device of claim 1, wherein the elongate body comprises a wall having a first portion and a second portion, the first portion having a higher material stiffness than the second portion to deter the elongate body from inwardly collapsing.

34. The nasopharyngeal airway device of claim 33, wherein the first portion is positioned adjacent a transverse midline of the elongate body.

35. The nasopharyngeal airway device of claim 33, wherein the first portion is positioned adjacent a vertical midline of the elongate body.

36. The nasopharyngeal airway device of claim 33, wherein at least one of a) the first portion has a Shore A hardness of approximately 50 A and the second portion has a Shore A hardness of approximately 40 A, b) the first portion has a Shore A hardness of between approximately 10 A and approximately 70 A and the second portion has a Shore A hardness of between approximately 10 A and approximately 70 A, or c) the first portion has a Shore A hardness of between approximately 30 A and approximately 60 A and the second portion has a Shore A hardness of between approximately 30 A and approximately 60 A.

37-38. (canceled)

39. The nasopharyngeal airway device of claim 33, wherein the elongate body comprises a first longitudinal portion, a second longitudinal portion, and a third longitudinal portion, the second longitudinal portion positioned between the first longitudinal portion and the third longitudinal portion, the first longitudinal portion and the third longitudinal portion each comprising the first portion and the second portion, the second longitudinal portion having an inner surface that defines the channel and has a spiral protrusion.

40-41. (canceled)

42. The nasopharyngeal airway device of claim 1, wherein the elongate body has an inner surface that defines the channel and has a spiral protrusion.

43. The nasopharyngeal airway device of claim 1, further comprising a bridal securement component securely coupled to the elongate body, the bridal securement component to interact with a nasal bridal to secure the nasopharyngeal airway device within a nostril of an individual.

44-50. (canceled)
